(19) United States
(12) Patent Application Publication
(10) Pub. No.: US 2019/0162720 A1
(43) Pub. Date: May 30, 2019

(54) SOLID PHASE SUPPORT FOR PROTEIN ANALYSIS AND METHOD FOR PRODUCING SAME

(71) Applicant: KAO CORPORATION, Tokyo (JP)

(72) Inventors: Tsuyoshi OBA, Chiba-shi (JP); Sayaka NOJI, Utsunomiya-shi (JP)

(73) Assignee: KAO CORPORATION, Tokyo (JP)

(21) Appl. No.: 16/314,477

(22) PCT Filed: Jul. 3, 2017
(86) PCT No.: PCT/JP2017/024368

§ 371 Date: Dec. 31, 2018

(30) Foreign Application Priority Data
Jul. 4, 2016 (JP) 2016-132569

(51) Int. Cl. G01N 33/543; G01N 33/536; B01J 20/286; B01J 20/26

(52) U.S. Cl. CPC G01N 33/54306; B01J 20/262; B01J 20/286; G01N 33/536

(57) ABSTRACT

A solid phase support capable of orienting the recognition site of an antibody for detection toward a measurement sample side and of immobilizing the antibody in a measurement area is provided. The solid phase support for protein analysis, wherein a first antibody is immobilized on a surface of a substrate, and a second antibody having an affinity to a substance to be analyzed is linked to the first antibody via protein A or G or a variant thereof is provided.

### FIELD OF INVENTION

The present invention relates to a solid phase support for protein analysis and a method for producing the same.

### BACKGROUND OF INVENTION

In the field of biotechnology, medicine, or clinical examination, biomolecules and derivatives thereof are often detected and quantified using specific interaction between molecules, such as antigen-antibody reaction or enzyme reaction.

For example, immunoassays such as enzyme-linked immunosorbent assay (ELISA) are known as methods for analyzing proteins such as physiologically active substances. In the immunoassays, the presence or absence of protein interaction with an antibody immobilized in a measurement area is indirectly measured using an enzyme-labeled or fluorescence-labeled secondary antibody and detecting the enzyme reaction or fluorescence. In addition, recently, a technique for directly detecting intermolecular interaction of a protein interacting with an immobilized antibody, by measuring change in refractive index using surface plasmon resonance (SPR) or measuring a property of varying (decreasing) the resonance frequency by quartz crystal microbalance (QCM), is also widely used. Thus, for analysis using an antibody, it is important to immobilize the antibody in a measurement area.

In general, immobilization of a protein such as an antibody is classified roughly into physical immobilization (adsorption) and chemical immobilization. Immobilization of an antibody by a chemical binding method has a problem of requiring a large number of steps for immobilizing the antibody and an increased number of reaction reagents and further has a problem of being unable to immobilize the antibody molecule at a specific site of the molecule and resulting in random orientation of the immobilized antibody, whereby not all the immobilized antibody can function.

In addition, recently, a higher sensitive immunoassay is required, and it is believed that it is important to ensure the amount of antibody immobilized for enhancing the intensity of the signal that can be detected and also to suppress non-specific adsorption for controlling the orientation of the antibody and reducing noise. Regarding the control of orientation of an antibody, it has been reported that the orientation of an antibody can be controlled by immobilizing the antibody via protein A immobilized on a base material by using specific binding between protein A and IgG (Non Patent Literatures 1 and 2) and a method using a fusion protein of a polystyrene-affinity peptide called a PS-tag and an antibody fragment (Non Patent Literature 3).

In addition, for example, a method for immobilizing biotinylated protein G to a biotinylated solid phase support via streptavidin (Patent Literature 1) and a method for suppressing non-specific adsorption and improving the amount of protein immobilized using a material such as polyethylene glycol (PEG) (Non Patent Literature 4) have been reported.

However, the techniques for using, for example, a PS-tag, are not convenient because of need of, for example, synthesis of a fusion protein and preparation of affinity peptides for the respective substrate surfaces. Although a substrate surface modified with, for example, polyethylene glycol is suppressed in non-specific adsorption, introduction of, for example, an antibody for detection is difficult to reduce the amount of the antibody immobilized for detection, resulting in difficulty to increase the detection sensitivity, which is problematic.
(Patent Literature 1) International Publication No. WO 2014/132692
(Non Patent Literature 1) Analytica Chimica Acta, 2012, 728, 64-68
(Non Patent Literature 2) Anal. Chem., 2011, 83, 1969-1976
(Non Patent Literature 3) Anal. Bioanal. Chem., 2009, 395, 759-765
(Non Patent Literature 4) Anal. Chem., 2005, 77, 1075-1080

### SUMMARY OF INVENTION

The present invention relates to the following aspects 1) to 4):

1) A solid phase support for protein analysis, wherein a first antibody is immobilized on a surface of a substrate, and a second antibody having an affinity to a substance to be analyzed is linked to the first antibody via protein A or G or a variant thereof;

2) A method for producing a solid phase support for protein analysis, comprising binding a first antibody to a surface of a substrate, then reacting and binding protein A or G or a variant thereof to the first antibody, and then reacting and binding a second antibody having an affinity to a substance to be analyzed to the protein A or G or variant thereof;

3) A solid phase support for capturing immunoglobulin, wherein a first antibody is immobilized on a surface of a substrate, and protein A or G or a variant thereof binds to the antibody; and

4) A method for capturing or quantifying immunoglobulin, using the solid phase support according to the aspect 3).

### BRIEF DESCRIPTION OF DRAWING

FIG. 1 is a schematic view of a solid phase support of the present invention.

### DETAILED DESCRIPTION OF INVENTION

The present invention provides a solid phase support capable of regiospecifically immobilizing an antibody for detection that binds to a substance to be analyzed at the Fc portion of the antibody molecule and capable of immobilizing the antibody with controlled orientation in a measurement area.

The present inventors have investigated construction of a solid phase interface for protein analysis capable of controlling the orientation of an antibody for detection and, as a result, have found that a solid phase support in which a second antibody (antibody for detection) is immobilized on a surface of a substrate with controlled orientation can be easily and efficiently produced by regiospecifically linking a first antibody immobilized on the surface of the substrate to the second antibody having an affinity to a substance to be analyzed via protein A or G or a variant thereof.

According to the present invention, a solid phase support in which a sufficient amount of an antibody for detection is regiospecifically immobilized at the Fc portion of the molecule thereof with controlled orientation in a measurement area can be provided. In addition, according to the present invention, synthesis of a fusion protein and so on is not necessary, and a sufficient amount of an antibody for detection can be immobilized even on a substrate surface which is modified with, for example, polyethylene glycol and having high performance of suppressing non-specific adsorption.

The solid phase support of the present invention can be used in the technical field related to analysis of protein and capture, quantification, or purification of immunoglobulin, for example, a chromatography support, a protein sensor, an immunoaffinity support, an antibody array, affinity analysis, and affinity separation.

In the solid phase support for protein analysis of the present invention, a first antibody is immobilized on a surface of a substrate, and a second antibody having an affinity to a substance to be analyzed is linked to the first antibody via protein A or G or a variant thereof.

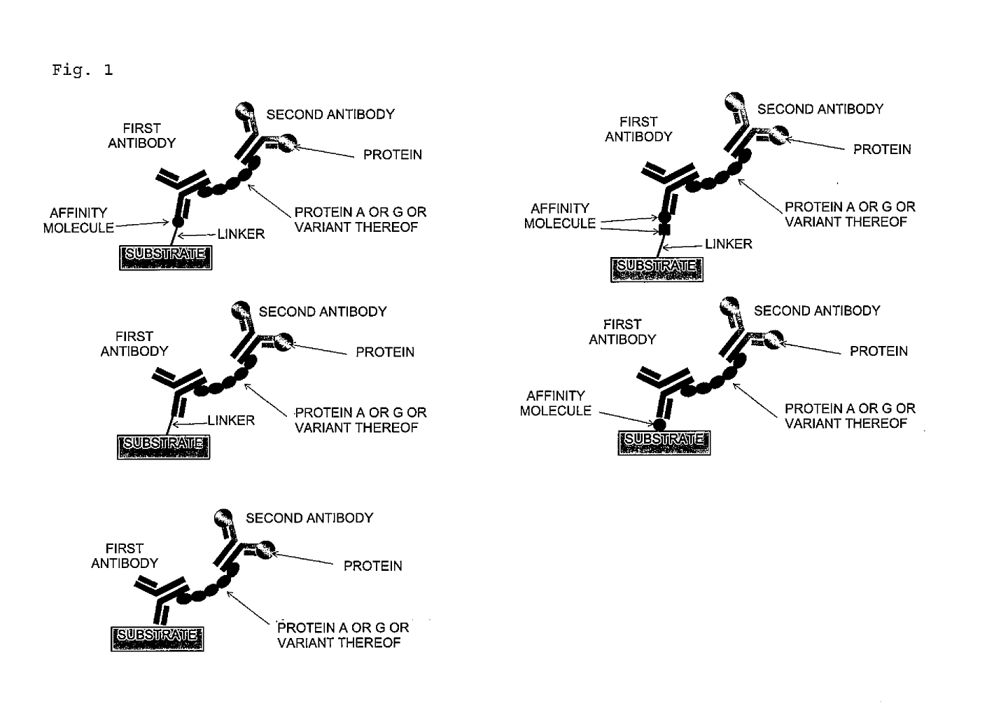

A schematic view thereof is shown in FIG. 1.

In the present invention, the term “protein analysis” encompasses the concepts of separation/purification, detection, and measurement of a protein. The solid phase support of the present invention will now be described.

### <Support Substrate>

In the solid phase support of the present invention, the substrate (supporting medium) may have any shape and may be made of any material as long as a first antibody can bind to its surface by physical binding.

Here, the substrate may have any insoluble shape that can immobilize the first antibody, such as particles, monoliths, films, fibers, hollow fibers, plates, sheets, and magnetic beads.

As the material of forming the substrate having such a shape, for example, plastics such as polyethylene, polypropylene, polystyrene, polymethacrylate, and polyvinyl alcohol or hydrogel; natural materials such as agarose, dextran, cellulose, chitosan, and latex; inorganic materials such as silica, glass, and ceramic; and metal materials such as gold, alumina, and silver can be widely used.

In addition, it is possible to appropriately introduce a functional group such as an amino group, carboxy group, or a vinyl group into a surface of the substrate using a known surface treatment technique.

In addition, a molecule (linker) capable of appropriately securing a distance between the first antibody and the substrate may bind to the surface of the substrate by reason, for example, of ease of immobilization of the first antibody and from the viewpoint of orientation control and reactivity of the second antibody. The molecule that can function as a linker is usually selected according to, for example, the charge characteristics of the surface of the solid phase support, and suitable examples thereof include thiol derivatives, such as alkanethiol forming a self-assembled monolayer (SAM); hydrophilic polymers containing polyethylene glycol chains (PEG chains); and MPC polymers (polymers of 2-methacryloyloxyethyl phosphorylcholine).

Among these examples, a substrate of a metal, such as gold, silver, aluminum, copper, or platinum, on which a self-assembled monolayer (SAM) is formed or to which PEG binds is preferred as the substrate of the solid phase support of the present invention.

Here, the self-assembled monolayer means an organic monolayer with uniform molecule orientation formed on a surface of a substrate by, when reactive organic molecules such as alkanethiol are contacted with an appropriate substrate material and are left to stand, chemically reacting the organic molecules with the substrate material to chemically adsorb the organic molecules on the substrate surface, and at the same time tightly gathering the adsorbed molecules through the interaction between the organic molecules. The self-assembled monolayer is widely used in the fields of, for example, surface plasmon resonance (SPR), surface plasmon-field enhanced fluorescence spectroscopy (SPFS), and quartz crystal microbalance (QCM).

In the present invention, examples of the organic molecules forming a self-assembled monolayer that can be used on a substrate include compounds represented by the formula (1): X—R [where, X represents SH—(CH₂)ₙ— or SH—(CH₂)ₙ—(O—CH₂—CH₂)ₘ—(OCH₂)ₗ—, and R represents a hydrogen atom, a carboxy group, a hydroxyl group, a hydroxymethyl group, an amino group, an aminomethyl group, an aldehyde group, an amide group (—CONH₂ group), a phosphonic acid group, a sulfo group, a quaternary ammonium group such as a trimethylamino group, a vinyl group, an acetylene group, an azido group, or a sulfobetaine group, where n represents an integer of 2 to 18, m represents an integer of 1 to 18, and l represents 0 or 1].

As the above-mentioned organic molecules, a disulfide formed by oxidative binding in a single organic molecule or between different organic molecules can also be used. Derivatives in which the thiol group of each organic molecule is acetylated can also be used.

In formula (1), X is preferably SH—(CH₂)ₙ—(O—CH₂—CH₂)ₘ—(OCH₂)ₗ—, where n is preferably 2 to 16, more preferably 6 to 16; m is preferably 1 to 12, more preferably 3 to 6. R is preferably hydrogen, a carboxy group, an aminomethyl group, or a hydroxymethyl group.

Examples of the organic molecules forming the self-assembled monolayer include 1-carboxyundecanethiol, 10-carboxy-1-decanethiol, 11-hydroxy-1-undecanethiol, and carboxy-EG₆-undecanethiol (another name: 20-(11-mercaptoundecanyloxy)-3,6,9,12,15,18-hexaoxaeicosanoic acid).

In the present invention, the organic molecules forming the self-assembled monolayer may be used alone, but may be also used as a mixture of two or more types. In such a case, a combination of carboxyalkanethiol and hydroxyalkanethiol and a combination of carboxy polyethyleneoxy alkanethiol and hydroxy polyethyleneoxy alkanethiol are preferred. For example, a mixture of 10-carboxy-1-decanethiol and 11-hydroxy-1-undecanethiol and a mixture of carboxy alkanethiol or hydroxy alkanethiol containing a PEG chain [—(O—CH₂—CH₂)ₘ— (OCH₂)ₙ— (where, m is 1 to 18 and n is 0 or 1)] between a carboxy group or a hydroxy group and an alkyl group are preferred. Specifically, examples of the mixture include a mixture of carboxy-EG₆-undecanethiol and hydroxy-EG₆-undecanethiol (another name: 29-mercapto-3,6,9,12,15,18-hexaoxanonacosan-1-ol) at an arbitrary mixing ratio.

In the hydrophilic polymer containing a PEG chain, the PEG chain [—(CH₂—CH₂—O)ₙ— (n is an integer indicating the degree of polymerization)] preferably has a number-average molecular weight or weight-average molecular weight of 200 or more, more preferably 500 or more, and preferably 30,000 or less, more preferably 10,000 or less, more preferably 5,000 or less. The number-average molecular weight or the weight-average molecular weight is preferably from 200 to 30,000, more preferably from 500 to 10,000, more preferably from 500 to 5,000.

The PEG chain in the hydrophilic polymer is preferably linear but may be a branched chain. In addition, a plurality of PEG polymers having different chain lengths may be introduced on the surface of the substrate as linker molecules.

The hydrophilic polymer comprising the PEG chain can have at least one functional group for chemically introducing the molecule to a substrate at an end of the PEG chain. Examples of the functional group include an amino group, a carboxyl group, a thiol group, an epoxy group, an aldehyde group, a maleimide group, an azido group, a cyano group, an active ester group (e.g., a succinimidyloxycarbonyl group, 1H-benzotriazol-1-yloxycarbonyl group, a pentafluorophenyloxycarbonyl group, or a paranitrophenyloxycarbonyl group), a (1H-imidazol-1-yl)carbonyl group, and a halogenated carbonyl group (a carbonyl chloride group, a carbonyl fluoride group, a carbonyl bromide group, or a carbonyl iodide group).

In addition, the PEG chain can have a molecule having an affinity to a first antibody described later or a functional group (for example, a carboxyl group, an amino group, a maleimide group, or an epoxy group) for binding the molecule in addition to an alkoxy group such as a methoxy group at another site other than the above-mentioned end (for example, the other end).

In addition, in the case of introducing a hydrophilic polymer containing a PEG chain to a gold substrate, it is preferable to use a hydrophilic polymer (PEG thiol reagent) having a thiol group at one end of the PEG chain and an alkoxy group such as a methoxy group, a carboxyl group, or an amino group, or the like at the other end, and the polymer can be introduced to the gold substrate by being contacted with the gold substrate. It is also possible to introduce a plurality of hydrophilic polymers containing such PEG chains having thiol groups at their ends.

In addition, it is preferable to introduce a molecule having an affinity to a first antibody to the substrate surface of the present invention (including the above-described surface into which a functional group or a linker is introduced) for making it easy to immobilize the first antibody by physical binding.

Here, examples of the molecule having an affinity to a first antibody include antibody binding molecules. The antibody binding molecule is a molecule including an antigen to the antibody, and examples thereof specifically include molecules shown below:

biotin, digoxigenin, and derivatives thereof;

avidin, streptavidin, NeutrAvidin, and complexes thereof with biotin or its derivative;

peptide such as GFP (Green Fluorescent Protein), RFP (Red Fluorescent Protein), histidine-tag (6×His-tag), DDDDK-tag, HA-tag (peptide derived from Human Influenza Hemagglutinin (HA)), Myc-tag (peptide derived from c-Myc protein), v5-tag (peptide derived from Simian Virus 5), S-tag (peptide derived from Pancreatic RNase A), E-tag, T7-tag (peptide derived from capsid protein of bacteriophage T7), and VSV-G-tag;

Glutathione-S-transferase (GST), Luciferase, Renilla Luciferase, β-galactosidase, Maltose Binding Protein (MBP), Thioredoxin (Trx), Chitin Binding Domain (CBD), and Calmodulin Binding Protein;

proteins forming non-specific adsorption preventing layers, such as albumin, casein, globulin, gelatin, skimmed milk, fibronectin, and lysozyme; and

PEG derivatives having various chain lengths.

Examples of the biotin derivative include biotin ethylenediamine monoamide and N-(5-aminopentyl)biotinamide.

Examples of a method of chemically introducing the molecule having an affinity to a first antibody to a surface of a substrate include a method by chemically binding the molecule to an arbitrary functional group present in a linker on the substrate or the substrate surface or to an arbitrary functional group appropriately introduced (for example, a carboxy group, an amino group, a thiol group, a maleimide group, or an epoxy group) or spacer molecule. In an example of the method, a carboxyl group on a substrate or on a linker is appropriately activated using, for example, water-soluble carbodiimide (WSC) and sodium N-hydroxysulfosuccinimide (NHS), and a solution in which a molecule having an affinity to the first antibody is dissolved or dispersed is then adhered thereto. In this case, the concentration of the molecule having an affinity to the first antibody is preferably from 0.0001 to 10 mM, more preferably about from 0.001 to 1 mM from the viewpoint of introduction efficiency of the molecule having an affinity to the first antibody.

Examples of the spacer molecule include PEG derivatives, and the chain length thereof is, in —(CH₂—CH₂—O—)ₙ—, n preferably denoting from 1 to 24, more preferably from 2 to 24, more preferably from 4 to 24.

Examples of the PEG derivative include H₂N—(CH₂—CH₂—O)ₙ—CH₂—CH₂—COOH, H₂N—(CH₂—CH₂—O)ₙ—CH₂—CH₂—NH₂, HOOC—(CH₂—CH₂—O)ₙ—CH₂—CH₂—COOH, HOOC—CH₂—CH₂—COO—(CH₂—CH₂—O)ₙ—CO—CH₂—CH₂—COOH, and derivatives thereof.

In the case of using a PEG derivative as the spacer molecule, the PEG derivative can be introduced by reacting the PEG derivative with the surface substituent (for example, a carboxyl group, an amino group, a hydroxyl group, a maleimide group, or a thiol group) on the substrate or the linker via an ester bond, an amide bond, an ether bond, or a thioether bond.

Alternatively, a molecule having an affinity to a first antibody is previously introduced into a molecule that can serve as a linker, and the molecule may bind to a substrate. For example, a thiol derivative or a PEG thiol reagent having a thiol group at one end and biotin at the other end bound by an ester bond or an amide bond is immobilized on a gold substrate through the thiol group side; or a monobiotinylated PEG derivative (for example, a salt of biotin-NH₂—CH₂—CH₂—NH—OC—(CH₂—CH₂—O—)ₙ—CH₂CH₂—NH₂ [n is 2 to 23]), which is a spacer molecule having biotin introduced therein, is previously introduced to a linker molecule, and the resultant linker is used as the thiol derivative or the like and is introduced to a substrate.

In the case of using a protein known as a protein forming a non-specific adsorption preventing layer (so-called blocking agent), such as albumin, casein, globulin, gelatin, or skimmed milk, as the molecule having an affinity to a first antibody, the protein can be introduced on a substrate surface by treating the substrate surface with a solution containing the protein and a buffer.

### <First Antibody>

In the present invention, a first antibody is immobilized on a surface of a substrate.

Here, the antibody to be used is not limited as long as it may be any antibody having a constant region (Fc domain) that binds to a molecule of the substrate surface and binds to protein A or G or a variant thereof. For example, antiserum prepared from serum of an animal immunized with an antigen that is recognized by the antibody, an immunoglobulin fraction purified from antiserum, or a monoclonal antibody or a polyclonal antibody produced by cell fusion using spleen cells of an animal immunized with the antigen can be used. Furthermore, a half antibody produced by cleavage of the disulfide bond between the heavy chains (H chains) may be used.

Immobilization of a first antibody on a substrate surface is performed by affinity binding between the first antibody and a molecule on the substrate surface, van der Waals force, Coulomb force, or physical binding other than covalent bond such as hydrogen bond. Examples thereof include binding include binding by physical interaction with a self-assembled monolayer on the substrate surface, affinity binding to PEG on the substrate surface, and binding to an affinity molecule introduced on the substrate surface or a linker (such as a self-assembled monolayer or PEG) on the substrate surface.

For example, in the case where a first antibody forms affinity binding together with PEG, an anti-PEG antibody is used as the first antibody, and in the case where a first antibody forms affinity binding together with a low molecular weight compound such as biotin or digoxigenin or a protein such as albumin or streptavidin, an antibody against the molecule, for example, an anti-biotin antibody, an anti-digoxigenin antibody, an anti-albumin antibody, or an anti-streptavidin antibody is used as the first antibody.

In the case of using an anti-PEG antibody, either an antibody forming affinity binding together with the substituent such as a methoxy group at an end of the PEG chain or an antibody forming affinity binding together with the PEG chain can be used as the anti-PEG antibody.

As the antibodies, commercially available antibodies may be purchased and used, or in the case of using a monoclonal antibody, immunization is performed with a corresponding immunogen, then, a hybridoma is produced by cell fusion, and the antibody secreted from the hybridoma may be purified and used. For example, a biotinylated antibody can be produced by reacting biotin or a derivative thereof with a known carrier protein. For example, an anti-biotin antibody can be produced by using biotinylated denatured Ovalbumin as an antigen, and an anti-PEG antibody can be produced by using an antigen prepared by conjugating a PEG derivative to Keyhole Limpet Haemocyanin (KLH) or the like.

When a first antibody is reacted with a molecule on a substrate surface by standing or solution feeding, the concentration of the antibody is not particularly limited and is preferably from 1 to 1,000 μg/mL.

In the case of physically binding a first antibody on a self-assembled monolayer, for example, the binding can be achieved by applying a solution containing the antibody to the self-assembled monolayer and performing standing or solution feeding at 0° C. to 50° C. for several minutes to several hours.

### <Protein A or G or Variant Thereof>

In the present invention, protein A or G or a variant thereof binds to both the first antibody and the second antibody.

Protein A is a protein being a cell wall component that is generated by Staphylococcus aureus, recognizes the constant regions (Fc domains) of immunoglobulin G, immunoglobulin A, and immunoglobulin M and has an activity (antibody binding activity) of binding to them by noncovalent bonds. Protein A is a multidomain membrane protein consisting of a plurality of domains, and a part of extracellular domains among them shows the activity (antibody binding activity) of binding to a protein having the Fc region of immunoglobulin G. For example, in the case of protein A derived from strain NCTC8325, five domains, E, D, A, B, and C, show the antibody binding activity. These domains each have a size of slightly less than 60 amino acids, and high homology is observed among the amino acid sequences thereof. In addition, it is known that each domain alone isolated by cleaving protein A maintains the antibody binding activity. In contrast, the Z domain is a modified protein in which such as mutation is introduced that Ala at position 1 and Gly at position 29 in the B domain are replaced with Val and Ala, respectively (Tashiro M, Montelione G T, (1995) Structures of bacterial immunoglobulin-binding domains and their complexes with immunoglobulins, Curr. Opin. Struct. Biol., 5, 471-481), and has improved alkali resistance and binding capacity compared with B domain.

Protein G is also a protein produced by Streptococcus and has an activity of specifically binding to the Fc region of immunoglobulin G. Protein G is also a multidomain membrane protein consisting of a plurality of domains, and a part of extracellular domains among them shows the activity (antibody binding activity) of binding to the Fc region of immunoglobulin. For example, in the case of protein G derived from strain G148, three domains, B1, B2, and B3 (also denoted as C1, C2, and C3 domains in some cases), show the antibody binding activity. Protein G of strain GX7805 has three antibody-binding domains, and Protein G of strain GX7809 has two antibody-binding domains. These domains each have a size of slightly less than 60 amino acids, and high homology is observed among the amino acid sequences thereof. In addition, it is known that each domain alone isolated by cleaving protein G maintains the antibody binding activity (Gallagher T, Alexander P, Bryan P, Gilliland G L, (1994) Two crystal structures of the B1 immunoglobulin-binding domain of streptococcal protein G and comparison with NMR, Biochemistry, 19, 4721-4729).

In order to enhance the antibody binding activity of protein A or G, recombinant protein A or recombinant protein G (referred to as “variant”) having modification by protein engineering have been created, and use of such a variant is preferred in the present invention.

Examples of the variant include, in addition to the above-described Z domain of protein A, a variant having the immunoglobulin binding domains of protein A or protein G and the total number of the domains of two or more, and a fusion variant having the immunoglobulin binding domains of protein A and protein G and the total number of the domains of 2 or more, preferably 2 to 20, more preferably 2 to 12. More specifically, examples of such variants include genetic recombinants in which regions other than the immunoglobulin binding domains are removed: protein G having two or three domains in total (manufactured by Thermo Fisher Scientific Inc., manufactured by BioVision Incorporated), protein A having five domains (manufactured by Thermo Fisher Scientific Inc., manufactured by BioVision Incorporated), and fusion protein A/G having the immunoglobulin binding domains of protein A and protein G and having six or eight domains in total (manufactured by Thermo Fisher Scientific Inc., manufactured by BioVision Incorporated).

The concentration of protein A or G or a variant thereof when it is reacted with the first antibody on the substrate surface is not particularly limited and is preferably from 1 to 1,000 μg/mL.

### <Second Antibody>

The second antibody is an antibody that binds to a compound to be analyzed and is immobilized by binding to the first antibody via protein A or G or a variant thereof.

The second antibody is not particularly limited as long as it may specifically react with a compound to be measured. For example, antiserum prepared from serum of an animal immunized with a compound that is recognized by the antibody, an immunoglobulin fraction purified from antiserum, or a monoclonal antibody or a polyclonal antibody produced by cell fusion using spleen cells of an animal immunized with the compound can be used. A modification may be made as in the case of a chimeric antibody. Furthermore, a half antibody produced by cleavage of the disulfide bond between the heavy chains (H chains) may be used.

As the second antibody, an antibody from any animal species can be used. For example, an antibody derived from, for example, a monkey, a mouse, a rat, a rabbit, chicken, a goat, sheep, or a guinea pig, specifically, mouse IgG, rat IgG, rabbit IgG, goat IgG, sheep IgG, or the like can be used, and the second antibody may be either a polyclonal antibody or a monoclonal antibody.

Any subclass of the antibody can be used as long as it can bind to protein A or G or a variant thereof.

The concentration of the second antibody when it is reacted with protein A or G or a variant thereof on the substrate surface is not particularly limited and is preferably from 1 to 1,000 μg/mL.

### <Production of Solid Phase Support>

In the production of the solid phase support of the present invention, first of all, a molecule having an affinity to a first antibody, a linker molecule having an affinity to the first antibody, or a linker molecule bound to a compound having an affinity to the first antibody is immobilized on a substrate. In the case where the substrate surface itself has an affinity to the first antibody, such immunization is not necessary. Thereafter, as long as the first antibody is immobilized on the substrate surface and a second antibody will be immobilized to the first antibody via protein A or G or a variant thereof, the binding may be performed in any order: a first antibody, protein A or G or a variant thereof, and a second antibody may be immobilized in this order on a substrate surface; a complex of protein A or G or a variant thereof and a second antibody may bind to a first antibody immobilized on a substrate surface; or a complex of a first antibody, protein A or G or a variant thereof, and a second antibody is previously prepared and may be immobilized on a substrate surface.

Preferred is a method including 1) binding a first antibody to a substrate surface, then 2) reacting and binding protein A or G or a variant thereof to the first antibody, and then 3) reacting and binding a second antibody to the protein A or G or variant thereof.

Here, the binding between the substrate surface and the first antibody, the binding between the first antibody and protein A or G or a variant thereof, and the binding between the protein A or G or variant thereof and the second antibody are all physical binding based on intermolecular interaction other than covalent bonds. Accordingly, the immobilization of each molecule of the first antibody, protein A or G or a variant thereof, and the second antibody can be performed by, for example, dripping solutions prepared by dissolving the respective molecules in a running buffer (for example, DPBS (Dulbecco's Phosphate Buffered Saline)) on the substrate, or by, when Biacore T200 is used, feeding the solutions on the substrate at a flow rate of 1 to 30 μL/min per one flow cell (length: 2.9 mm, width: 0.5 mm, height: 0.04 mm) for 5 to 60 minutes, alternatively by dipping the measurement substrate in the solutions of the respective molecules.

Thus, according to the present invention, a second antibody is regiospecifically immobilized on a substrate surface at the Fc region in the molecular structure by a noncovalent bond due to physical interaction based on intermolecular interaction other than covalent bonds by using protein A or G or a variant thereof. Accordingly, the invention is useful from the viewpoint of controlling the orientation of the antibody molecules, compared with a method partially employing a chemical binding method, which results in random orientation of the antibody.

After the immobilization procedure, the portion of the solid phase where the antibody is not immobilized may be blocked with a blocking agent, such as BSA (bovine serum albumin), Block Ace, skimmed milk, or casein. Furthermore, together with the blocking agent, or after removal of the blocking agent, a stabilizer for stabilizing the immobilization, for example, a synthetic or natural macromolecule such as polyethylene glycol or a polysaccharide, a surfactant, or a commercially available Immunoassay Stabilizer (ABI Co., Ltd.), can be added. In addition, in the case of using a protein interaction analyzer or the like, a surfactant may be added to each solution that is used in the immobilization procedure, in order to reduce the loss of each protein due to adhesion to the flow channel, etc. of each solution to be used.

The solid phase support produced during the process of the immobilization procedure in which a first antibody is immobilized on a substrate surface and protein A or G or a variant thereof binds to the first antibody can be used as a solid phase support for purification or capturing or quantitation of an antibody molecule including a Fc portion capable of binding to protein A or G or a variant thereof, specifically immunoglobulin, preferably IgG portion.

In order to produce the solid phase support for protein analysis described above, a kit containing at least a substrate, a first antibody, protein A or G or a variant thereof, and a second antibody may be used. By using the kit, it is possible to regiospecifically immobilize a sufficient amount of an antibody for detection at the Fc portion of the molecule in a measurement area with controlled orientation.

Regarding the above-described embodiments, the present invention further discloses the following aspects:

<1> A solid phase support for protein analysis, wherein a first antibody is immobilized on a surface of a substrate, and a second antibody having an affinity to a substance to be analyzed is linked to the first antibody via protein A or G or a variant thereof;

<2> The solid phase support according to aspect <1>, wherein the first antibody is immobilized via a molecule having an affinity to the antibody and introduced on the surface of the substrate;

<3> The solid phase support according to aspect <2>, wherein the molecule having an affinity to the first antibody is biotin, digoxigenin, or a derivative thereof;

<4> The solid phase support according to aspect <2>, wherein the molecule having an affinity to the first antibody is a protein selected from the group consisting of albumin, casein, globulin, gelatin, skimmed milk, fibronectin, and lysozyme;

<5> The solid phase support according to aspect <2>, wherein the molecule having an affinity to the first antibody is avidin, streptavidin, or NeutrAvidin or a complex thereof with biotin or its derivative;

<6> The solid phase support according to any one of aspects <1> to <5>, wherein the surface of the substrate is a surface on which a hydrophilic polymer comprising a polyethylene glycol chain is introduced;

<7> The solid phase support according to aspect <6>, wherein the hydrophilic polymer comprising a polyethylene glycol chain is a hydrophilic polymer comprising a PEG chain having an average molecular weight of 500 to 5,000;

<8> The solid phase support according to any one of aspects <1> to <5>, wherein the surface of the substrate is a surface on which a self-assembled monolayer is formed;

<9> The solid phase support according to aspect <8>, wherein the self-assembled monolayer is an alkanethiol derivative;

<10> The solid phase support according to any one of aspects <1> to <9>, wherein the variant of protein A or G is a variant having immunoglobulin binding domains of protein A or protein G and the total number of the domains of two or more, or a fusion variant having immunoglobulin binding domains of protein A and protein G and the total number of the domains of two or more;

<11> The solid phase support according to aspect <10>, wherein the total number of the domains of the variant of protein A or G is from 2 to 12;

<12> A method for producing a solid phase support for protein analysis, comprising binding a first antibody to a surface of a substrate, then reacting and binding protein A or G or a variant thereof to the first antibody, and then reacting and binding a second antibody having an affinity to a substance to be analyzed to the protein A or G or variant thereof;

<13> A kit for producing the solid phase support according to any one of aspects <1> to <11>, comprising at least a substrate, a first antibody, protein A or G or a variant thereof, and a second antibody;

<14> A solid phase support for capturing or quantifying immunoglobulin, wherein a first antibody is immobilized on a surface of a substrate, and protein A or G or a variant thereof binds to the antibody;

<15> The solid phase support according to aspect <14>, wherein the immunoglobulin is IgG; and

<16> A method for capturing or quantifying immunoglobulin, comprising using the solid phase support according to aspect <14> or <15>.

### EXAMPLES

### Example 1

### (1) Formation of SAM

A sensor chip (GE Healthcare, SIA Kit Au, BR-1004-05), which is used in Biacore T-200, was immersed in a 100 μM ethanol solution (10 mL) of carboxy-EG₆-undecanethiol (20-(11-mercaptoundecanyloxy)-3,6,9,12,15,18-hexaoxaeicosanoic acid, Dojindo Laboratories, C445) and shaken at 25° C. overnight to form a self-assembled monolayer (SAM) on the gold surface of the chip. The sensor chip after immersing and shaking was washed with ethanol and Milli-Q water and was then dried with nitrogen.

### (2) Immobilization of Biotin Derivative

On the sensor chip provided with the SAM, 300 μL of a solution prepared by mixing MES buffer (2-morpholinoethanesulfonic acid, monohydrate, 80 mM, pH: 5.60) solutions of 1-ethyl-3-(3-dimethylaminopropyl)carbodiimide hydrochloride (Peptide Institute, Inc., 4 mg/100 μL) and sodium N-hydroxysulfosuccinimide (s-NHS, Wako Pure Chemical Corporation, 2 mg/mL) at a ratio of 1:1 was dripped and was left to stand for 10 minutes. Subsequently, after washing with Milli-Q water, the dripping operation was repeated again to convert the carboxyl group of the SAM surface to an active ester group. Subsequently, the chip was immersed in a 1 μM DMF solution (10 mL) in which N-(5-aminopentyl)biotinamide (trifluoroacetate salt) was neutralized with equimolar triethylamine and shaken at 25° C. for 2 hours. Subsequently, the chip was washed with Milli-Q water, and 300 μL of a 1 M ethanolamine solution was then dripped on the chip surface for 10 minutes to perform hydrolysis treatment of the unreacted active ester. This dripping operation was repeated twice. Subsequently, the chip was washed with Dulbecco's phosphate-buffered saline (Life Technology Inc., DPBS, 14190-144, pH: 7.0-7.3), dried with nitrogen and then used for SPR measurement. The SPR measurement was performed using Biacore T200 at 25° C.

### (3) Immobilization of Anti-Biotin Antibody

Immobilization of an anti-biotin antibody on the biotinylated surface of the sensor chip was performed by feeding a 100 μg/mL solution in which the anti-biotin antibody (Abcam plc., ab53494, Rabbit polyclonal) was dissolved in DPBS as a running buffer on the sensor chip at a flow rate of 10 μL/min for 15 minutes and then feeding the running buffer again for 15 minutes. The amount of the anti-biotin antibody immobilized was 3,376.4 RU.

### (4) Immobilization of Protein A/G

Immobilization of protein A/G (Pierce 21186, Recombinant Protein A/G from E. coli, six domains) on the sensor chip surface on which the anti-biotin antibody was immobilized was performed by feeding a 100 μg/mL solution in which the protein A/G was dissolved in DPBS as a running buffer on the sensor chip at a flow rate of 10 μL/min for 15 minutes and then feeding the running buffer again for 15 minutes. The amount of the protein A/G immobilized was 558.4 RU.

### (5) Immobilization of Anti-Adiponectin Antibody

Immobilization of an anti-adiponectin antibody (R&D Systems, Inc., Human Adiponectin/Acrp30 Antibody, Monoclonal Mouse IgG_{2B}, MAB10651) on the sensor chip surface on which the anti-biotin antibody and protein A/G were immobilized was performed by feeding a 100 μg/mL solution in which the anti-adiponectin antibody was dissolved in DPBS as a running buffer on the sensor chip at a flow rate of 10 μL/min for 20 minutes and then feeding the running buffer again for 40 minutes. The amount of the anti-adiponectin antibody immobilized was 1,104.9 RU. The results are shown in Table 1.

### (6) BSA Blocking

Blocking of the anti-adiponectin antibody-immobilized sensor chip surface with Bovine Serum Albumin (Sigma-Aldrich Co. LLC, BSA, A3059-10G) was performed by feeding a PBS solution (1 mg/mL) in which the BSA was dissolved in DPBS as a running buffer on the sensor chip at a flow rate of 10 μL/min for 20 minutes and then feeding the running buffer again for 40 minutes. The amount of the BSA adsorbed was 674.7 RU.

### (7) Amount of Antigen (Adiponectin) Bound

The SPR measurement of the amount of antigen bound on the sensor chip surface prepared by the operations (1) to (6) was also performed using Biacore T200 at 25° C. As a running buffer, a DPBS solution containing BSA at a concentration of 1 mg/mL and containing 0.05% (V/W) Tween 20 was used. Adiponectin (Enzo Life Sciences, Inc. ALX-522-063-C050, Human, Recombinant, HEK293 cells) was also dissolved in the running buffer and used. On the sensor chip, the 1.0 μg/mL solution was fed at a flow rate of 10 μL/min for 20 minutes, and then the running buffer was fed again for 20 minutes, and the amount of antigen bound was measured. The amount of antigen bound was determined by correcting with reference data. In the process of immobilizing the anti-adiponectin antibody, a detection interface was formed by immobilizing an anti-biotin antibody instead of the anti-adiponectin antibody, and the amount of antigen bound at the interface was subtracted as the reference data from a measured value for correction. The amount of antigen bound after the correction was 336.2 RU. The results are shown in Table 1.

### Example 2

### (1) Formation of SAM

A SAM was formed according to the method of (1) in Example.

### (2) Immobilization of Biotin Derivative

A biotin derivative, N-(5-aminopentyl)biotinamide (trifluoroacetate salt), was immobilized according to the method of (2) in Example 1. However, the concentration of the biotin derivative solution was 10 μM.

### (3) Immobilization of Anti-Biotin Antibody

An anti-biotin antibody was immobilized according to the method of (3) in Example 1. The amount of the anti-biotin antibody immobilized was 6,318.9 RU.

### (4) Immobilization of Protein A/G

Protein A/G was immobilized according to the method of (4) in Example 1. The amount of the protein A/G immobilized was 718.3 RU.

### (5) Immobilization of Anti-Adiponectin Antibody

An anti-adiponectin antibody was immobilized according to the method of (5) in Example 1. The amount of the anti-adiponectin antibody immobilized was 934.8 RU. The results are shown in Table 1.

### (6) BSA Blocking

BSA blocking was performed according to the method of (6) in Example 1. The amount of BSA adsorbed was 360.8 RU.

### (7) Amount of Antigen (Adiponectin) Bound

The amount of antigen bound was measured according to the method of (7) in Example 1. The amount of antigen bound after the correction was 285.8 RU. The results are shown in Table 1.

### Example 3

### (1) Formation of Mixed SAM

A sensor chip (GE Healthcare, SIA Kit Au, BR-1004-05), which is used in Biacore T-200, was immersed in a solution mixture (10 mL) of a 500 μM ethanol solution of 10-carboxy-1-decanethiol (Dojindo Laboratories, C385) and a 500 μM ethanol solution of 11-hydroxy-1-undecanethiol (Dojindo Laboratories, H337) at a ratio of 1:9 and shaken at 25° C. for 10 minutes to form a self-assembled monolayer (SAM) on the gold surface of the chip. The sensor chip surface after immersion and shaking was washed with ethanol and Milli-Q water and was then dried with nitrogen.

### (2) Immobilization of Biotin Derivative

A biotin derivative, N-(5-aminopentyl)biotinamide (trifluoroacetate salt), was immobilized according to the method of (2) in Example 1. However, the concentration of the biotin derivative solution was 1 mM.

### (3) Immobilization of Anti-Biotin Antibody

An anti-biotin antibody was immobilized according to the method of (3) in Example 1. The amount of the anti-biotin antibody immobilized was 5,095.7 RU.

### (4) Immobilization of Protein A/G

Protein A/G was immobilized according to the method of (4) in Example 1. The amount of the protein A/G immobilized was 889.9 RU.

### (5) Immobilization of Anti-Adiponectin Antibody

An anti-adiponectin antibody was immobilized according to the method of (5) in Example 1. The amount of the anti-adiponectin antibody immobilized was 1,609.7 RU. The results are shown in Table 1.

### (6) BSA Blocking

BSA blocking was performed according to the method of (6) in Example 1. The amount of BSA adsorbed was 946.4 RU.

### (7) Amount of Antigen (Adiponectin) Bound

The amount of antigen bound was measured according to the method of (7) in Example 1. The amount of antigen bound after the correction was 275.7 RU. The results are shown in Table 1.

### Example 4

### (1) Formation of Mixed SAM

A mixed SAM was formed according to the method of (1) in Example 3.

However, a solution mixture prepared by mixing solutions of thiol reagents, 10-carboxy-1-decanethiol and 11-hydroxy-1-undecanethiol, at a ratio of 1:99 was used.

### (2) Immobilization of Biotin Derivative

A biotin derivative was immobilized according to the method of (2) in Example 3.

### (3) Immobilization of Anti-Biotin Antibody

An anti-biotin antibody was immobilized according to the method of (3) in Example 1. The amount of the anti-biotin antibody immobilized was 4,892.8 RU.

### (4) Immobilization of Protein A/G

Protein A/G was immobilized according to the method of (4) in Example 1. The amount of the protein A/G immobilized was 884.2 RU.

### (5) Immobilization of Anti-Adiponectin Antibody

An anti-adiponectin antibody was immobilized according to the method of (5) in Example 1. The amount of the anti-adiponectin antibody immobilized was 1,623.1 RU. The results are shown in Table 1.

### (6) BSA Blocking

BSA blocking was performed according to the method of (6) in Example 1. The amount of BSA adsorbed was 907.6 RU.

### (7) Amount of Antigen (Adiponectin) Bound

The amount of antigen bound was measured according to the method of (7) in Example 1. The amount of antigen bound after the correction was 320.8 RU. The results are shown in Table 1.

### Example 5

### (1) Formation of Mixed SAM

A mixed SAM was formed according to the method of (1) in Example 3.

However, a solution mixture prepared by mixing solutions of 10-carboxy-1-decanethiol and 11-hydroxy-1-undecanethiol at a ratio of 1:1999 was used.

### (2) Immobilization of Biotin Derivative

A biotin derivative was immobilized according to the method of (2) in Example 3.

### (3) Immobilization of Anti-Biotin Antibody

An anti-biotin antibody was immobilized according to the method of (3) in Example 1. The amount of the anti-biotin antibody immobilized was 4,791.6 RU.

### (4) Immobilization of Protein A/G

Protein A/G was immobilized according to the method of (4) in Example 1. The amount of the protein A/G immobilized was 889.5 RU.

### (5) Immobilization of Anti-Adiponectin Antibody

An anti-adiponectin antibody was immobilized according to the method of (5) in Example 1. The amount of the anti-adiponectin antibody immobilized was 1,556.1 RU. The results are shown in Table 1.

### (6) BSA Blocking

BSA blocking was performed according to (6) in Example 1. The amount of BSA adsorbed was 1,184.1 RU.

### (7) Amount of Antigen (Adiponectin) Bound

The amount of antigen bound was measured according to the method of (7) in Example 1. The amount of antigen bound after the correction was 258.5 RU. The results are shown in Table 1.

### Comparative Example 1

### (1) Formation of SAM

A SAM was formed according to the method of (1) in Example 1. However, the volume of the ethanol solution of the thiol reagent was 5 mL.

### (2) Immobilization of Anti-Adiponectin Antibody

The steps after the immobilization of an anti-adiponectin antibody were performed using Biacore T200 at a flow rate of 10 μL/min at 25° C. The sensor chip provided with a SAM was set on Biacore T200, a solution prepared by mixing MES buffer solutions of 1-ethyl-3-(3-dimethylaminopropyl)carbodiimide hydrochloride (4 mg/100 μL) and sodium N-hydroxysulfosuccinimide (2 mg/mL) at a ratio of 1:1 was fed for 7 minutes, and a solution in which 50 μg/mL anti-adiponectin antibody was dissolved in PBS as a running buffer was then fed for 20 minutes. A 1 M ethanolamine aqueous solution was further fed to the chip for 7 minutes to thereby perform hydrolysis treatment of the unreacted active ester, and the running buffer was then fed again for 5 minutes. The amount of the anti-adiponectin antibody immobilized was 429.1 RU. The results are shown in Table 1.

### (3) BSA Blocking

Blocking with BSA of the sensor chip surface on which the anti-adiponectin antibody was immobilized was performed by feeding a PBS solution (1 mg/L) in which BSA was dissolved in DPBS as a running buffer on the sensor chip at a flow rate of 10 μL/min for 15 minutes and then feeding the running buffer again for 5 minutes. The amount of BSA adsorbed was 112.9 RU.

### (4) Amount of Antigen (Adiponectin) Bound

The SPR measurement of the amount of antigen bound on the sensor chip surface prepared by the operations (1) to (3) was performed using Biacore T200 at 25° C. As a running buffer, a DPBS solution containing BSA at a concentration of 1 mg/mL and containing 0.05% (V/W) Tween 20 was used. Adiponectin was also dissolved in the solution and used, and the amount of antigen bound was measured by feeding the 1.0 μg/mL solution on the sensor chip at a flow rate of 10 μL/min for 15 minutes and feeding the running buffer again for 10 minutes. The amount of antigen bound was determined by correcting with reference data. In the process of immobilizing the anti-adiponectin antibody, a detection interface was formed by immobilizing an anti-interleukin 6 (IL6) antibody (R&D Systems, Inc., Human IL-6 Antibody, Monoclonal Mouse IgG_{2B}, MAB2061) instead of the anti-adiponectin antibody, and the amount of antigen bound at the interface was subtracted as the reference data from a measured value for correction. The amount of antigen bound after the correction was 100.7 RU. The results are shown in Table 1.

TABLE 1

| | | | | | Amount of | |
|---|---|---|---|---|---|---|
| | | | | Protein A/G | second antibody | Amount of |
| | | | First antibody | (number of | immobilized | antigen bound |
| Example | Linker | Affinity molecule | (Concentration: μg/mL) | domains) | (RU) | (RU) |
| Example 1 | EG₆ | Biotin | Anti-biotin antibody | A/G (6) | 1104.9 | 336.2 |
| | | (1 μM) | (100) | | | |
| Example 2 | EG₆ | Biotin | Anti-biotin antibody | A/G (6) | 934.8 | 285.8 |
| | | (10 μM) | (100) | | | |
| Example 3 | C10 + C11 | Biotin | Anti-biotin antibody | A/G (6) | 1609.7 | 275.7 |
| | Thiol mixing ratio | (1 mM) | (100) | | | |
| | COOH:OH = 1:9 | | | | | |
| Example 4 | C10 + C11 | Biotin | Anti-biotin antibody | A/G (6) | 1623.1 | 320.8 |
| | Thiol mixing ratio | (1 mM) | (100) | | | |
| | COOH:OH = 1:99 | | | | | |
| Example 5 | C10 + C11 | Biotin | Anti-biotin antibody | A/G (6) | 1556.1 | 258.5 |
| | Thiol mixing ratio | (1 mM) | (100) | | | |
| | COOH:OH = 1:999 | | | | | |
| Comparative | EG₆ | — | — | — | 429.1 | 100.7 |
| Example 1 | | | | | | |

EG₆: carboxy-EG₆-undecanethiol
C10 + C11: mixture of 10-carboxy-1-decanethiol (C10) and 11-hydroxy-1-undecanethiol (C11)
Second antibody: anti-adiponectin antibody
RU: unit (resonance unit) in measurement with Biacore T200

### Example 6

### (1) Formation of SAM

A SAM was formed according to the method of (1) in Example 1.

### (2) Immobilization of Biotin Derivative

A biotin derivative, N-(5-aminopentyl)biotinamide (trifluoroacetate salt), was immobilized according to the method of (2) in Example 1. However, the concentration of the biotin derivative solution was 100 μM.

### (3) Immobilization of Anti-Biotin Antibody

Immobilization of an anti-biotin antibody on the biotinylated surface of the sensor chip was performed at 25° C. by feeding a 5.0 μg/mL solution in which an anti-biotin monoclonal antibody (Abcam plc., ab36406, Mouse monoclonal) was dissolved in DPBS as a running buffer on the sensor chip at a flow rate of 10 μL/min for 30 minutes and then feeding the running buffer again for 20 minutes. The amount of the anti-biotin antibody immobilized was 4,493.2 RU.

### (4) Immobilization of Protein G

Immobilization of protein G (BioVision Incorporated, 6501, recombinant protein G, three domains) on the sensor chip surface on which the anti-biotin antibody was immobilized was performed at 25° C. by feeding a 40 μg/mL solution in which protein G was dissolved in DPBS as a running buffer on the sensor chip at a flow rate of 10 μL/min for 30 minutes and then feeding the running buffer again for 30 minutes. The amount of the protein G immobilized was 841.4 RU.

### (5) Immobilization of Anti-CRP Antibody

Immobilization of an anti-CRP antibody (R&D Systems, Inc., Human C-Reactive protein Antibody, Monoclonal Mouse IgG_{2B}, MAB17071-500) on the sensor chip surface on which the anti-biotin antibody and protein G were immobilized was performed at 25° C. by feeding a 100 μg/mL solution in which the anti-CRP antibody was dissolved in DPBS as a running buffer on the sensor chip at a flow rate of 10 μL/min for 30 minutes and then feeding the running buffer again for 30 minutes. The amount of the anti-CRP antibody immobilized was 2,013.1 RU. The results are shown in Table 2.

### (6) BSA Blocking

Blocking with BSA of the sensor chip surface on which the anti-CRP antibody was immobilized was performed by feeding a PBS solution (1 mg/mL) in which BSA was dissolved in DPBS as a running buffer on the sensor chip at a flow rate of 10 μL/min for 20 minutes and then feeding the running buffer again for 10 minutes. The amount of BSA adsorbed was −194.3 RU.

### (7) Amount of Antigen (CRP) Bound

The SPR measurement of the amount of antigen bound on the sensor chip surface prepared by the operations (1) to (6) was performed using Biacore T200 at 25° C. As a running buffer, a DPBS solution containing BSA at a concentration of 1 mg/mL and containing 0.05% (V/W) Tween 20 was used. C-Reactive protein (CRP, R&D Systems, Inc., 1707-CR-200, Mouse myeloma cell line) was dissolved in the running buffer and used, and the amount of antigen bound was measured by feeding the 1.0 μg/mL solution on the sensor chip at a flow rate of 10 μL/min for 30 minutes and then feeding the running buffer again for 30 minutes. At the reference interface formed by immobilizing an anti-interleukin 6 antibody instead of the anti-CRP antibody in the above (5), no antigen-antibody reaction was observed even if the CRP solution was fed. Accordingly, it is believed that in this step, significant physical adsorption of the antigen to the detection interface did not occur, and the amount of CRP bound was 512.6 RU. The results are shown in Table 2.

### Example 7

### (1) Formation of SAM

A SAM was formed according to the method of (1) in Example 1.

### (2) Immobilization of Biotin Derivative

A biotin derivative, N-(5-aminopentyl)biotinamide (trifluoroacetate salt), was immobilized according to the method of (2) in Example 6.

### (3) Immobilization of Anti-Biotin Antibody

An anti-biotin antibody was immobilized according to the method of (3) in Example 6. The amount of the anti-biotin antibody immobilized was 4,631.5 RU.

### (4) Immobilization of Protein A/G

Immobilization of protein A/G (Pierce 21186, Recombinant Protein A/G from E. coli, six domains) on the sensor chip surface on which the anti-biotin antibody was immobilized was performed at 25° C. by feeding a 25 μg/mL solution in which the protein A/G was dissolved in DPBS as a running buffer on the sensor chip at a flow rate of 10 μL/min for 30 minutes and then feeding the running buffer again for 30 minutes. The amount of the protein A/G immobilized was 622.4 RU.

### (5) Immobilization of Anti-CRP Antibody

Immobilization was performed according to the method of (5) in Example 6. The amount of the anti-CRP antibody immobilized was 2,303.4 RU. The results are shown in Table 2.

### (6) BSA Blocking

Blocking was performed according to the method of (6) in Example 6. The amount of BSA adsorbed was −19.4 RU.

### (7) Amount of Antigen (CRP) Bound

The amount of antigen bound was measured according to the method of (7) in Example 6. The amount of CRP bound was 422.4 RU. The results are shown in Table 2.

### Example 8

### (1) Formation of SAM

A SAM was formed according to the method of (1) in Example 1.

### (2) Immobilization of Biotin Derivative

A biotin derivative, N-(5-aminopentyl)biotinamide (trifluoroacetate salt), was immobilized according to the method of (2) in Example 6.

### (3) Immobilization of Anti-Biotin Antibody

An anti-biotin antibody was immobilized according to the method of (3) in Example 6. The amount of the anti-biotin antibody immobilized was 4,702.1 RU.

### (4) Immobilization of Protein A/G

Immobilization of protein A/G (BioVision Incorporate, 6502, Recombinant Protein A/G from E. coli, eight domains) on the sensor chip surface on which the anti-biotin antibody was immobilized was performed at 25° C. by feeding a 30 μg/mL solution in which the protein A/G was dissolved in DPBS as a running buffer on the sensor chip at a flow rate of 10 μL/min for 30 minutes and then feeding the running buffer again for 30 minutes. The amount of the protein A/G immobilized was 868.2 RU.

### (5) Immobilization of Anti-CRP Antibody

Immobilization was performed according to the method of (5) in Example 6. The amount of the anti-CRP antibody immobilized was 3,455.9 RU. The results are shown in Table 2.

### (6) BSA Blocking

Blocking was performed according to the method of (6) in Example 6. The amount of BSA adsorbed was −106.2 RU.

### (7) Amount of Antigen (CRP) Bound

The amount of antigen bound was measured according to the method of (7) in Example 6. The amount of CRP bound was 744.6 RU. The results are shown in Table 2.

### Comparative Example 2

### (1) Formation of SAM

Formation of a SAM was performed according to the method of (1) in Comparative Example 1.

### (2) Immobilization of Anti-CRP Antibody

The steps after the immobilization of an anti-CRP antibody were performed using Biacore T200 at a flow rate of 10 μL/min at 25° C. The sensor chip provided with a SAM was set on Biacore T200, a solution prepared by mixing MES buffer solutions of EDC (4 mg/100 μL) and s-NHS (2 mg/mL) at a ratio of 1:1 was fed for 7 minutes, and a solution in which 50 μg/mL anti-CRP antibody was dissolved in PBS as a running buffer was fed for 20 minutes. A 1 M ethanolamine aqueous solution was further fed to the chip for 7 minutes to thereby perform hydrolysis treatment of the unreacted active ester, and the running buffer was then fed again for 5 minutes. The amount of the anti-CRP antibody immobilized was 1,564.4 RU. The results are shown in Table 2.

### (3) BSA Blocking

The sensor chip surface on which the anti-CRP antibody was immobilized was blocked with BSA by feeding a PBS solution (1 mg/mL) in which BSA was dissolved in PBS as a running buffer on the sensor chip at a flow rate of 10 μL/min for 15 minutes and then feeding the running buffer again for 5 minutes. The amount of BSA adsorbed was 73.6 RU.

### (4) Amount of Antigen Bound

The SPR measurement of the amount of antigen bound on the sensor chip surface prepared by the operations (1) to (3) was performed using Biacore T200 at 25° C. As a running buffer, a DPBS solution containing BSA at a concentration of 1 mg/mL and containing 0.05% (V/W) Tween 20 was used. C-Reactive protein (CRP, R&D Systems, Inc., 1707-CR-200, Mouse myeloma cell line) was also dissolved in the solution and used, and the amount of antigen bound was measured by feeding the 1.0 μg/mL solution on the sensor chip at a flow rate of 10 μL/min for 15 minutes and feeding the running buffer again for 10 minutes. The antigen-antibody reaction amount was determined by correcting with reference data. In the process of immobilizing the anti-CRP antibody, a detection interface was formed by immobilizing an anti-interleukin 6 (IL6) antibody instead of the anti-CRP antibody, and the antigen-antibody reaction amount at the interface was subtracted as the reference data from a measured value for correction. The amount of CRP bound after the correction was 284.2 RU. The results are shown in Table 2.

TABLE 2

| | | | | Protein A- | Amount of | |
|---|---|---|---|---|---|---|
| | | | | related | second | |
| | | | First antibody | protein | antibody | Amount of |
| | | Affinity | (Concentration: | (number of | immobilized | antigen bound |
| | Linker | molecule | μg/mL) | domains) | (RU) | (RU) |
| Example 6 | EG₆ | Biotin | Anti-biotin | G (3) | 2013.1 | 512.6 |
| | | | antibody | | | |
| | | | (5.0) | | | |
| Example 7 | EG₆ | Biotin | Anti-biotin | A/G (6) | 2303.4 | 422.4 |
| | | | antibody | | | |
| | | | (5.0) | | | |
| Example 8 | EG₆ | Biotin | Anti-biotin | A/G (8) | 3455.9 | 744.6 |
| | | | antibody | | | |
| | | | (5.0) | | | |
| Comparative | EG₆ | — | — | — | 1564.4 | 284.2 |
| Example 2 | | | | | | |

EG₆: carboxy-EG₆-undecanethiol
Second antibody: anti-CRP antibody
RU: unit (resonance unit) in measurement with Biacore T200

### Example 9

(1) Formation of mPEGylated Interface

Formation of a methoxy polyethylene glycol-coated interface (mPEGylated interface) was performed using Biacore T-200 at 37° C. As a running buffer, a solution prepared by adding 14.61 g of sodium chloride to Dulbecco's phosphate-buffered saline (Life Technology Inc., DPBS, 14190-144, pH: 7.0-7.3, 250 g) was used. A running buffer-dissolving solution prepared by dissolving mPEG thiol 5 k (manufactured by Creative PEGWorks, mPEG-SH, MW: 5,000, PLS-604, 1 mg/mL) in the running buffer was fed on a sensor chip at a flow rate of 15 μL/min for 20 minutes, followed by feeding the running buffer for 10 minutes, a 0.05 N NaOH solution for 1 minute, the running buffer for 3 minutes, the 0.05 N NaOH solution for 1 minute, and then the running buffer for 11 minutes at a flow rate of 15 μL/min. This series of operations was repeated twice to immobilize the mPEG thiol 5 k on the sensor chip surface. The amount of the mPEG thiol 5 k immobilized was 2,940.1 RU in total. Subsequently, mPEG thiol 2 k (manufactured by Creative PEGWorks, mPEG-SH, MW: 2,000, PLS-605) was further immobilized on the same chip under the same conditions. The amount of the mPEG thiol 2 k immobilized was 65.6 RU in total.

### (2) Immobilization of Anti-mPEG Antibody

Immobilization of an anti-mPEG antibody on the mPEGylated surface of the sensor chip was performed at 25° C. by feeding a 10.0 μg/mL solution in which an anti-mPEG monoclonal antibody (Abcam plc., ab51257, Mouse monoclonal, Anti-Polyethylene glycol antibody [PEG-B-47]) was dissolved in DPBS as a running buffer on the sensor chip at a flow rate of 10 μL/min for 30 minutes and then feeding the running buffer again for 20 minutes. The amount of the anti-mPEG antibody immobilized was 3,906.9 RU.

### (3) Immobilization of Protein A/G

Immobilization of protein A/G (Pierce 21186, Recombinant Protein A/G from E. coli, six domains) on the sensor chip surface on which the anti-mPEG antibody was immobilized was performed at 25° C. by feeding a 101 μg/mL solution in which the protein A/G was dissolved in DPBS as a running buffer on the sensor chip at a flow rate of 10 μL/min for 30 minutes and then feeding the running buffer again for 30 minutes. The amount of the protein A/G immobilized was 636.8 RU.

### (4) Immobilization of Anti-CRP Antibody

Immobilization of an anti-CRP antibody (R&D Systems, Inc., Human C-Reactive protein Antibody, Monoclonal Mouse IgG_{2B}) on the sensor chip surface on which the anti-mPEG antibody and protein A/G were immobilized was performed at 25° C. by feeding a 100 μg/mL solution in which the anti-CRP antibody was dissolved in DPBS as a running buffer on the sensor chip at a flow rate of 10 μL/min for 30 minutes and then feeding the running buffer again for 30 minutes. The amount of the anti-CRP antibody immobilized was 2,841.9 RU. The results are shown in Table 3.

### (5) BSA Blocking

Blocking was performed according to the method of (6) in Example 6. The amount of BSA adsorbed was −38.5 RU.

### (6) Amount of Antigen (CRP) Bound

The amount of antigen bound was measured according to the method of (7) in Example 6. The amount of CRP bound was 407.9 RU. The results are shown in Table 3.

### Example 10

(1) Formation of mPEGylated Interface

Formation of an mPEGylated interface was performed using Biacore T-200 at 37° C. As a running buffer, a solution prepared by adding 14.61 g of sodium chloride to Dulbecco's phosphate-buffered saline (Life Technology Inc., DPBS, 14190-144, pH: 7.0-7.3, 250 g) was used. A running buffer-dissolving solution prepared by dissolving mPEG thiol 2 k (manufactured by Creative PEGWorks, mPEG-SH, MW: 2,000, PLS-605, 1 mg/mL) in the running buffer was fed on a sensor chip at a flow rate of 15 μL/min for 20 minutes, followed by feeding the running buffer for 20 minutes, a 0.05 N NaOH solution for 1 minute, the running buffer for 3 minutes, the 0.05 N NaOH solution for 1 minute, and then the running buffer for 11 minutes at a flow rate of 15 μL/min. This series of operations was repeated for five times to immobilize the mPEG thiol 2 k on the sensor chip surface. The amount of the mPEG thiol 2 k immobilized was 1,959.7 RU in total. Subsequently, poly(ethylene glycol) methyl ether thiol 800 (mPEG thiol 800, manufactured by Sigma-Aldrich Co. LLC, 729108, average Mn: 800, 1 mg/mL) was further immobilized on the same chip by repeating the operations under the same conditions for five times. The amount of the mPEG thiol 800 immobilized was 90.6 RU in total.

### (2) Immobilization of Anti-mPEG Antibody

Immobilization of an anti-mPEG antibody on the mPEGylated surface of the sensor chip was performed at 25° C. by feeding a 5.0 μg/ml, solution in which an anti-mPEG antibody (Abcam plc., ab51257, Mouse monoclonal, Anti-Polyethylene glycol antibody [PEG-B-47]) was dissolved in DPBS as a running buffer on the sensor chip at a flow rate of 10 μL/min for 30 minutes and then feeding the running buffer again for 30 minutes. The amount of the anti-mPEG antibody immobilized was 3,126.6 RU.

### (3) Immobilization of Protein A/G

Immobilization of protein A/G (Pierce 21186, Recombinant Protein A/G from E. coli, six domains) on the sensor chip surface on which the anti-mPEG antibody was immobilized was performed at 25° C. by feeding a 1,000 μg/mL solution in which the protein A/G was dissolved in DPBS as a running buffer on the sensor chip at a flow rate of 10 μL/min for 30 minutes and then feeding the running buffer again for 50 minutes. The amount of the protein A/G immobilized was 684.6 RU.

### (4) Immobilization of Anti-CRP Antibody

Immobilization was performed according to the method of (4) in Example 9. The amount of the anti-CRP antibody immobilized was 3,448.0 RU. The results are shown in Table 3.

### (5) BSA Blocking

The sensor chip surface on which the anti-CRP antibody was immobilized was blocked with BSA by feeding a PBS solution (1 mg/mL) in which BSA was dissolved in DPBS as a running buffer on the sensor chip at a flow rate of 10 μL/min for 20 minutes and then feeding the running buffer again for 30 minutes. The amount of BSA adsorbed was −119.3 RU.

### (6) Amount of Antigen (CRP) Bound

The amount of antigen bound was measured according to the method of (7) in Example 6. The amount of CRP bound was 662.8 RU. The results are shown in Table 3.

### Example 11

(1) Formation of mPEGylated Interface

Formation of mPEGylated interface was performed according to the method of (1) in Example 9. The amount of the mPEG thiol 5 k immobilized was 3,055.1 RU in total, and the amount of the mPEG thiol 2 k immobilized was 63.4 RU in total.

### (2) Immobilization of Anti-mPEG Antibody

Immobilization was performed according to the method of (2) in Example 9. The amount of the anti-mPEG antibody immobilized was 3,828.4 RU.

### (3) Immobilization of Protein A/G

Immobilization of protein A/G (BioVision Incorporated, 6502, Recombinant Protein A/G from E. coli, eight domains) on the sensor chip surface on which the anti-mPEG antibody was immobilized was performed at 25° C. by feeding a 119 μg/mL solution in which the protein A/G was dissolved in DPBS as a running buffer on the sensor chip at a flow rate of 10 μL/min for 30 minutes and then feeding the running buffer again for 30 minutes. The amount of the protein A/G immobilized was 819.8 RU.

### (4) Immobilization of Anti-CRP Antibody

Immobilization was performed according to the method of (4) in Example 9. The amount of the anti-CRP antibody immobilized was 3,207.8 RU. The results are shown in Table 3.

### (5) BSA Blocking

Blocking was performed according to the method of (6) in Example 6. The amount of BSA adsorbed was −119.8 RU.

### (6) Amount of Antigen (CRP) Bound

The amount of antigen bound was measured according to the method of (7) in Example 6. The amount of CRP bound was 536.1 RU. The results are shown in Table 3.

TABLE 3

| | | | | Amount of | |
|---|---|---|---|---|---|
| | | | | second | |
| | | First antibody | Protein A/G | antibody | Amount of |
| | | (Concentration: | (number of | immobilized | antigen bound |
| | Linker | μg/mL) | domains) | (RU) | (RU) |
| Example 9 | mPEG | Anti-mPEG | A/G (6) | 2841.9 | 407.9 |
| | (5k + 2k) | antibody | | | |
| | | (10.0) | | | |
| Example 10 | mPEG | Anti-mPEG | A/G (6) | 3448.0 | 662.8 |
| | (2k + 800) | antibody | | | |
| | | (5.0) | | | |
| Example 11 | mPEG | Anti-mPEG | A/G (8) | 3207.8 | 536.1 |
| | (5k + 2k) | antibody | | | |
| | | (10.0) | | | |

mPEG: methoxy polyethylene glycol
Second antibody: anti-CRP antibody
RU: unit (resonance unit) in measurement with Biacore T200

### Example 12

### (1) Formation of Biotin-PEGylated Interface

Formation of a biotin-PEGylated interface was performed using Biacore T-200 at 37° C. As a running buffer, a 1 M PBS solution (pH 7.4, 0.05 M) of sodium chloride was used. A running buffer-dissolving solution prepared by dissolving biotinylated PEG thiol 5 k (manufactured by Nanocs Inc., Biotin PEG Thiol, PG2-BNTH-5 k, 1 mg/mL) in the running buffer was fed on a sensor chip at a flow rate of 15 μL/min for 20 minutes, followed by feeding the running buffer for 10 minutes, a 0.05 N NaOH solution for 1 minute, the running buffer for 3 minutes, the 0.05 N NaOH solution for 1 minute, and then the running buffer for 11 minutes at a flow rate of 15 μL/min. This series of operations was repeated twice to immobilize the biotinylated PEG thiol 5 k on the sensor chip surface. The amount of the biotinylated PEG thiol 5 k immobilized was 1,781.2 RU in total. Subsequently, mPEG thiol 2 k (manufactured by Creative PEGWorks, mPEG-SH, MW: 2,000, PLS-605, 1 mg/mL) was further immobilized on the same chip under the same conditions. The amount of the mPEG thiol 2 k immobilized was 545.5 RU in total.

### (2) Immobilization of Anti-Biotin Antibody

Immobilization was performed according to the method of (3) in Example 6. However, the anti-biotin antibody solution used was a solution having a concentration of 10.0 μg/mL. The amount of the anti-biotin antibody immobilized was 3,604.5 RU.

### (3) Immobilization of Protein A/G

Immobilization of protein A/G (Pierce 21186, Recombinant Protein A/G from E. coli, six domains) on the sensor chip surface on which the anti-biotin antibody was immobilized was performed at 25° C. by feeding a 100 μg/mL solution in which the protein A/G was dissolved in DPBS as a running buffer on the sensor chip at a flow rate of 10 μL/min for 30 minutes and then feeding the running buffer again for 30 minutes. The amount of the protein A/G immobilized was 603.2 RU.

### (4) Immobilization of Anti-CRP Antibody

Immobilization was performed according to the method of (5) in Example 6. The amount of the anti-CRP antibody immobilized was 2,329.5 RU. The results are shown in Table 4.

### (5) BSA Blocking

Blocking was performed according to the method of (6) in Example 6. The amount of BSA adsorbed was −62.5 RU.

### (6) Amount of Antigen (CRP) Bound

The amount of antigen bound was measured according to the method of (7) in Example 6. The amount of CRP bound was 507.9 RU. The results are shown in Table 4.

### Example 13

### (1) Formation of Biotin-PEGylated Interface

Formation of a biotin-PEGylated interface was performed using Biacore T-200 at 37° C. As a running buffer, a 1 M PBS solution (pH 7.4, 0.05 M) of sodium chloride was used. A running buffer dissolving solution prepared by dissolving biotinylated PEG thiol 2 k (manufactured by Nanocs Inc., Biotin PEG Thiol, PG2-BNTH-2 k, 1 mg/mL) in the running buffer was fed on a sensor chip at a flow rate of 15 μL/min for 20 minutes, followed by feeding the running buffer for 10 minutes, a 0.05 N NaOH solution for 1 minute, the running buffer for 3 minutes, the 0.05 N NaOH solution for 1 minute, and then the running buffer for 11 minutes at a flow rate of 15 μL/min. This series of operations was repeated twice to immobilize the biotinylated PEG thiol 2 k on the sensor chip surface. The amount of the biotinylated PEG thiol 2 k immobilized was 2,121.6 RU in total. Subsequently, poly(ethylene glycol) methyl ether thiol 800 (mPEG thiol 800, manufactured by Sigma-Aldrich Co. LLC, 729108, average Mn: 800, 1 mg/mL) was further immobilized on the same chip under the same conditions. The amount of the mPEG thiol 800 immobilized was 533.1 RU in total.

### (2) Immobilization of Anti-Biotin Antibody

Immobilization was performed according to the method of (3) in Example 6. However, the anti-biotin antibody solution used was a solution having a concentration of 10.0 μg/mL. The amount of the anti-biotin antibody immobilized was 4,623.3 RU.

### (3) Immobilization of Protein A/G

Immobilization was performed according to the method of (3) in Example 12. The amount of the protein A/G immobilized was 827.5 RU.

### (4) Immobilization of Anti-CRP Antibody

Immobilization was performed according to the method of (5) in Example 6. The amount of the anti-CRP antibody immobilized was 2,889.3 RU. The results are shown in Table 4.

### (5) BSA Blocking

Blocking was performed according to the method of (6) in Example 6. The amount of BSA adsorbed was −48.9 RU.

### (6) Amount of Antigen (CRP) Bound

The amount of antigen bound was measured according to the method of (7) in Example 6. The amount of CRP bound was 559.6 RU. The results are shown in Table 4.

TABLE 4

| | | | | | Amount of | |
|---|---|---|---|---|---|---|
| | | | | | second | Amount of |
| | | | First antibody | Protein A/G | antibody | antigen |
| | | Affinity | (Concentration: | (number of | immobilized | bound |
| | Linker | molecule | μg/mL) | domains) | (RU) | (RU) |
| Example | PEG (5k + 2k) | Biotin | Anti-biotin antibody | A/G (6) | 2329.5 | 507.9 |
| 12 | | | (10.0) | | | |
| Example | PEG (2k + 800) | Biotin | Anti-biotin antibody | A/G (6) | 2889.3 | 559.6 |
| 13 | | | (10.0) | | | |

Second antibody: anti-CRP antibody
RU: unit (resonance unit) in measurement with Biacore T200

### Example 14

### (1) Formation of SAM

Formation of a SAM was performed according to the method of (1) in Example 1.

### (2) Immobilization of Biotin Derivative

Immobilization was performed according to the method of (2) in Example 6.

### (3) Immobilization of Streptavidin

Immobilization of streptavidin on the biotinylated surface of the sensor chip was performed at 25° C. by feeding a 50 μg/mL solution prepared by diluting a 10 mg/mL solution of streptavidin (Thermo Fisher Scientific Inc., 21122, 20 mM Potassium Phosphate, pH 6.5) with DPBS containing 0.05% (V/W) Tween 20 as a running buffer on the sensor chip at a flow rate of 10 μL/min for 30 minutes and then feeding the running buffer again for 10 minutes. The amount of the streptavidin immobilized was 1,367.6 RU.

### (4) Immobilization of Anti-Streptavidin Antibody

Immobilization of an anti-streptavidin antibody (R&D Systems, Inc., Streptavidin Antibody, Recombinant Monoclonal Rabbit IgG Clone #1220C, MAB9020) on the sensor chip surface on which streptavidin was immobilized was performed at 25° C. by feeding a 50 μg/mL solution in which the anti-streptavidin antibody was dissolved in DPBS containing 0.05% (V/W) Tween 20 as a running buffer on the sensor chip at a flow rate of 10 μL/min for 30 minutes and then feeding the running buffer again for 20 minutes. The amount of the anti-streptavidin antibody immobilized was 2,981.9 RU.

### (5) Immobilization of Protein A/G

Immobilization of protein A/G (Pierce 21186, Recombinant Protein A/G from E. coli, six domains) on the sensor chip surface on which the anti-streptavidin antibody was immobilized was performed at 25° C. by feeding a 100 μg/mL solution in which the protein A/G was dissolved in DPBS containing 0.05% (V/W) Tween 20 as a running buffer on the sensor chip at a flow rate of 10 μL/min for 30 minutes and then feeding the running buffer again for 30 minutes. The amount of the protein A/G immobilized was 715.7 RU.

### (6) Immobilization of Anti-CRP Antibody

Immobilization of an anti-CRP antibody (R&D Systems, Inc., Human C-Reactive protein Antibody, Monoclonal Mouse IgG_{2B}) on the sensor chip surface on which the anti-streptavidin antibody and protein A/G were immobilized was performed at 25° C. by feeding a 100 μg/mL solution in which the anti-CRP antibody was dissolved in DPBS containing 0.05% (V/W) Tween 20 as a running buffer on the sensor chip at a flow rate of 10 μL/min for 30 minutes and then feeding the running buffer again for 30 minutes. The amount of the anti-CRP antibody immobilized was 3,402.8 RU. The results are shown in Table 5.

### (7) Amount of Antigen (CRP) Bound

The SPR measurement of the amount of antigen bound on the sensor chip surface prepared by the operations (1) to (6) was performed using Biacore T200 at 25° C. C-Reactive protein (CRP, R&D Systems, Inc., 1707-CR-200, Mouse myeloma cell line) was dissolved in DPBS containing 0.05% (V/W) Tween 20 as a running buffer and used, and the amount of antigen bound was measured by feeding the 1.0 μg/mL solution on the sensor chip at a flow rate of 10 μL/min for 30 minutes and then feeding the running buffer again for 30 minutes. At the reference interface formed by immobilizing an anti-interleukin 6 antibody instead of the anti-CRP antibody in the above (5), no antigen-antibody reaction was observed even if the CRP solution was fed. Accordingly, it is believed that in this step, significant physical adsorption of the antigen to the detection interface did not occur, and the amount of CRP bound was 629.7 RU. The results are shown in Table 5.

TABLE 5

| | | | | | | Amount of | |
|---|---|---|---|---|---|---|---|
| | | | | | | second | Amount of |
| | | | | | Protein A/G | antibody | antigen |
| | | | | First antibody | (number of | immobilized | bound |
| | Linker | Affinity molecule | | (Concentration: μg/mL) | domains) | (RU) | (RU) |
| Example 14 | EG₆ | Biotin derivative | Streptavidin | Anti-streptavidin | A/G | 3402.8 | 629.7 |
| | | (100 μM) | (50 μg/mL) | antibody | (6) | | |
| | | | | (50) | | | |

EG₆: carboxy-EG₆-undecanethiol
Second antibody: anti-CRP antibody
RU: unit (resonance unit) in measurement with Biacore T200

### Example 15

### (1) Adsorption of BSA (Bovine Serum Albumin)

Measurement of the amount of BSA adsorbed on a sensor chip for measurement was performed using Biacore T-200 and was performed by feeding a 0.25% (W/V) solution in which BSA (Sigma-Aldrich Co. LLC, A3059) was dissolved in DPBS (Dulbecco's phosphate-buffered saline, Life Technology Inc., 14190-144, pH: 7.0-7.3) on a sensor chip for measurement (GE Healthcare, SIA Kit Au, BR-1004-05) at a flow rate of 10 μL/min for 20 minutes and then feeding the running buffer again for 10 minutes. The amount of BSA adsorbed was 2,117.6 RU.

### (2) Immobilization of Anti-BSA Antibody

Immobilization of an anti-BSA antibody on the sensor chip surface on which BSA was adsorbed was performed at 25° C. by feeding a 50 μg/mL solution in which a 1 mg/mL solution of an anti-BSA antibody (Bioss Inc., bs-0292R, polyclonal antibody) was diluted with DPBS containing 0.05% (V/W) Tween 20 as a running buffer on the sensor chip at a flow rate of 10 μL/min for 30 minutes and then feeding the running buffer again for 20 minutes. The amount of the anti-BSA antibody immobilized was 2,828.9 RU.

### (3) Immobilization of Protein A/G

Immobilization was performed according to the method of (5) in Example 14. The amount of the protein A/G immobilized was 602.3 RU.

### (4) Immobilization of Anti-CRP Antibody

Immobilization was performed according to the method of (6) in Example 14. The amount of the anti-CRP antibody immobilized was 2,802.4 RU. The results are shown in Table 6.

### (5) Amount of Antigen (CRP) Bound

The amount of antigen bound was measured according to the method of (7) in Example 14. The amount of CRP bound was 518.7 RU. The results are shown in Table 6.

### Example 16

### (1) Formation of SAM

A sensor chip (GE Healthcare, SIA Kit Au, BR-1004-05), which is used in Biacore T-200, was immersed in a 100 μM ethanol solution (10 mL) of 10-carboxy-1-decanethiol (Dojindo Laboratories, C385) and shaken at 25° C. overnight to form a self-assembled monolayer (SAM) on the gold surface of the substrate. The substrate after the formation was washed with ethanol and Milli-Q water and was then dried with nitrogen.

### (2) Adsorption of BSA (Bovine Serum Albumin)

Adsorption of BSA was performed according to the method of (1) in Example 15. However, the BSA solution used was a solution having a concentration of 0.005% (W/V). The amount of BSA adsorbed was 1,338.7 RU.

### (3) Immobilization of Anti-BSA Antibody

Immobilization was performed according to the method of (2) in Example 15. However, the anti-BSA solution used was a solution having a concentration of 25 μg/mL. The amount of the anti-BSA antibody immobilized was 2,557.9 RU.

### (4) Immobilization of Protein A/G

Immobilization was performed according to the method of (5) in Example 14. The amount of protein A/G immobilized was 496.9 RU.

### (5) Immobilization of Anti-CRP Antibody

Immobilization was performed according to the method of (6) in Example 14. The amount of the anti-CRP antibody immobilized was 2,815.8 RU. The results are shown in Table 6.

### (6) Amount of Antigen (CRP) Bound

The amount of antigen bound was measured according to the method of (7) in Example 14. The amount of CRP bound was 513.6 RU. The results are shown in Table 6.

### Example 17

### (1) Formation of SAM

Formation of a SAM was performed according to the method of (1) in Example 16.

### (2) Adsorption of BSA (Bovine Serum Albumin)

Adsorption of BSA was performed according to the method of (1) in Example 15. The amount of BSA adsorbed was 2,144.0 RU.

### (3) Immobilization of Anti-BSA Antibody

Immobilization was performed according to the method of (3) in Example 16. The amount of the anti-BSA antibody immobilized was 2,518.0 RU.

### (4) Immobilization of Protein A/G

Immobilization was performed according to the method of (5) in Example 14. The amount of the protein A/G immobilized was 506.6 RU.

### (5) Immobilization of Anti-CRP Antibody

Immobilization was performed according to the method of (6) in Example 14.

The amount of the anti-CRP antibody immobilized was 2,685.0 RU. The results are shown in Table 6.

### (6) Amount of Antigen Bound

The amount of antigen bound was measured according to the method of (7) in Example 14. The amount of CRP bound was 480.6 RU. The results are shown in Table 6.

TABLE 6

| | | | | | Amount of | |
|---|---|---|---|---|---|---|
| | | | | | second | Amount of |
| | | | First antibody | protein A/G | antibody | antigen |
| | | | (Concentration: | (number of | immobilized | bound |
| | Linker | Affinity molecule | μg/mL) | domains) | (RU) | (RU) |
| Example | — | BSA | Anti-BSA | A/G | 2802.4 | 518.7 |
| 15 | | (0.25 W/V %) | antibody | (6) | | |
| | | | (50) | | | |
| Example | C10 | BSA | Anti-BSA | A/G | 2815.8 | 513.6 |
| 16 | | (0.005 W/V %) | antibody | (6) | | |
| | | | (25) | | | |
| Example | C10 | BSA | Anti-BSA | A/G | 2685.0 | 480.6 |
| 17 | | (0.25 W/V %) | antibody | (6) | | |
| | | | (25) | | | |

Second antibody: anti-CRP antibody
C10: 10-carboxy-1-decanethiol
RU: unit (resonance unit) in measurement with Biacore T200)

1. A solid phase support for protein analysis, wherein a first antibody is immobilized on a surface of a substrate, and a second antibody having an affinity to a substance to be analyzed is linked to the first antibody via protein A or G or a variant thereof.

2. The solid phase support according to claim 1, wherein the first antibody is immobilized via a molecule having an affinity to the antibody and introduced to the surface of the substrate.

3. The solid phase support according to claim 2, wherein the molecule having an affinity to the first antibody is biotin, digoxigenin, or a derivative thereof.

4. The solid phase support according to claim 2, wherein the molecule having an affinity to the first antibody is a protein selected from the group consisting of albumin, casein, globulin, gelatin, skimmed milk, fibronectin, and lysozyme.

5. The solid phase support according to claim 2, wherein the molecule having an affinity to the first antibody is avidin, streptavidin, or NeutrAvidin or a complex thereof with biotin or its derivative.

6. The solid phase support according to claim 1, wherein the surface of the substrate is a surface on which a hydrophilic polymer comprising a polyethylene glycol chain is introduced.

7. The solid phase support according to claim 6, wherein the hydrophilic polymer comprising a polyethylene glycol chain is a hydrophilic polymer comprising a PEG chain having an average molecular weight of 500 to 5,000.

8. The solid phase support according to claim 1, wherein the surface of the substrate is a surface on which a self-assembled monolayer is formed.

9. The solid phase support according to claim 8, wherein the self-assembled monolayer is an alkanethiol derivative.

10. The solid phase support according to claim 1, wherein the variant of protein A or G is a variant having immunoglobulin binding domains of protein A or protein G and a total number of the domains of two or more, or a fusion variant having immunoglobulin binding domains of protein A and protein G and a total number of the domains of two or more.

11. The solid phase support according to claim 10, wherein the total number of the domains of the variant of protein A or G is 2 to 12.

12. A method for producing a solid phase support for protein analysis, comprising binding a first antibody to a surface of a substrate, then reacting and binding protein A or G or a variant thereof to the first antibody, and then reacting and binding a second antibody having an affinity to a substance to be analyzed to the protein A or G or variant thereof.

13. A kit for producing a solid phase support according to claim 1, comprising at least a substrate, a first antibody, protein A or G or a variant thereof, and a second antibody.

14. A solid phase support for capturing or quantifying immunoglobulin, wherein a first antibody is immobilized on a surface of a substrate, and protein A or G or a variant thereof binds to the antibody.

15. A method for capturing or quantifying immunoglobulin, comprising using the solid phase support according to claim 14.
